(19) United States
(12) Patent Application Publication
(10) Pub. No.: US 2024/0366556 A1
(43) Pub. Date: Nov. 7, 2024

(54) COMPOSITIONS COMPRISING PROANTHOCYANIDINS AND ZINC FOR TREATING AND/OR PREVENTING INFLAMMATION

(71) Applicant: ANIMORA INC., Québec (CA)

(72) Inventors: Jean-Philippe Cote, Québec (CA); Andree-Ann Adam, Québec (CA)

(21) Appl. No.: 18/579,054

(22) PCT Filed: Dec. 1, 2022
(86) PCT No.: PCT/CA2022/051759

§ 371 Date: Jan. 12, 2024

Related U.S. Application Data
(60) Provisional application No. 63/264,733, filed on Dec. 1, 2021.

(51) Int. Cl. A61K 31/353; A61K 9/00; A61K 9/06; A61K 33/30; A61K 36/45; A61K 47/36; A61K 47/44; A61K 47/46; A61P 29/00

(52) U.S. Cl. CPC A61K 31/353; A61K 9/0053; A61K 9/0056; A61K 9/0095; A61K 9/06; A61K 33/30; A61K 36/45; A61K 47/36; A61K 47/44; A61K 47/46; A61P 29/00

(57) ABSTRACT

A composition is provided for treating and/or preventing inflammation in a subject comprising A-type proanthocyanidin (PAC-A) and zinc. The composition can include between 0.001 wt % and 10 wt % of PAC-A, and between 0.01 wt % and 25 wt % of a zinc-containing compound. A method is provided for preventing and/or treating inflammation in a subject comprising administration of the composition comprising A-type proanthocyanidin (PAC-A) and zinc. The composition can be also used for the manufacture of a medicament for the treatment of inflammation. For example, the inflammation can be associated with an oral cavity, cutaneous membrane, or gastrointestinal tract. For example, a process is provided for producing an oral gel product, the process comprising providing a gel base; and admixing a zinc-containing compound and an A-type proanthocyanidin (PAC-A)-containing extract to the gel base to form the oral gel product.

### CROSS-REFERENCE TO RELATED APPLICATION

This application claims priority to International Canadian Patent Application No. PCT/CA2022/051759 filed on Dec. 1, 2022, which claims priority to U.S. Provisional Patent Application No. 63/264,733 filed on Dec. 1, 2021, the entire disclosures of which are incorporated herein by way of reference.

### TECHNICAL FIELD

The technical field generally relates to compositions for treating and/or preventing inflammation. Specifically, the technical field relates to compositions comprising proanthocyanidins and zinc for reducing inflammation in a subject.

### BACKGROUND

Inflammation is part of the body's biological response to harmful stimuli, such as bacteria or toxins. Inflammation, when left untreated can lead to serious inflammatory diseases or ailments within a variety of body tissues, such as epithelial, periodontal, or gastrointestinal tissues. For example, periodontal disease is an inflammatory disease of the gums and periodontal tissues that is caused by bacterial biofilms, such as gram-negative and anaerobic bacteria, that are in contact with the periodontal tissues. It is estimated that about 80% of dogs and 70% of cats have periodontal disease by the age of two and that severe periodontitis affects almost 20% of adult humans. If left untreated, the inflammation caused by periodontal disease can lead to a degradation of gums, degradation of the alveolar bone, tooth loss, and blood infection.

Conventional solutions used to reduce inflammation include synthetic drugs, such as non-steroidal anti-inflammatory drugs (NSAIDs) or steroids. However, these synthetic drugs can have adverse reactions with other drugs, such as blood thinners, and cannot be taken by people with heart disease and women who are pregnant. Polyphenols are known as a natural alternative for treating inflammation, such as the anti-inflammatory formulations in Canadian Patent No. 2.532.596. However, large quantities of natural extracts can be required to achieve an effective dosage of the polyphenols. Cranberries are a known source of polyphenols, such as described in Canadian Patent No. 2.730.716 and have been described for their antimicrobial and anti-bacterial properties in published international Patent Application WO 1996/028135.

Alternatives for treating inflammation of the oral cavity, such as gum inflammation, include the prevention of plaque and calculus or to reduce halitosis via growth of a bacterial film. Thus, the usual treatment of periodontal disease is directed at the bacterial infection rather than the inflammation of the tissue. However, reducing the bacterial infection can only partially reduce the gum inflammation; and can risk recurrence of bacteria infection when the immune system is engaged in fighting the inflammation.

There is therefore a need for improvement in compositions for preventing and/or treating inflammation and bacterial growth in a subject.

### SUMMARY

The present techniques allow manufacture and use of a composition including a combination of active ingredients having a synergistic effect toward reduction of inflammation in a subject. It has been found that the combination of the zinc and proanthocyanidin (PAC-A) renders a composition usable to treat inflammation.

In one aspect, there is provided a composition for treating and/or preventing inflammation in a subject comprising A-type proanthocyanidin (PAC-A) and zinc. For example, the composition can include at least 0.001 wt % of PAC-A with respect to a total weight of the composition. For example, the composition can include at least 0.01 wt % of PAC-A. For example, the composition can include at least 0.1 wt % of PAC-A. For example, the composition can include at least 1 wt % of PAC-A. For example, the composition can include at least 10 wt % of PAC-A. Optionally, the composition can include between 0.001 wt % and 10 wt % of PAC-A.

The composition can include a PAC-A containing extract for providing the PAC-A. The PAC-A containing extract can include between 5 wt % and 15 wt % of PAC-A with respect to a total weight of the PAC-A containing extract. For example, the composition can include between 0.05 wt % and 15 wt % of the PAC-A containing extract with respect to a total weight of the composition. Optionally, the composition can include between 0.05 wt % and 6 wt % of the PAC-A containing extract. Further optionally, the composition can include between 1 wt % and 3 wt % of the PAC-A containing extract.

In some implementations, the PAC-A containing extract can be isolated from a natural source. For example, the PAC-A containing extract can be provided as a PAC-A containing cranberry extract. In another example, the PAC-A containing extract can be an insoluble extract. In another example, the PAC-A containing extract can be a soluble extract. Optionally, the PAC-A containing extract can be a dried cranberry powder.

The composition can also include a zinc-containing compound for providing the zinc. For example, the zinc-containing compound can be a zinc salt. For example, the zinc salt can be selected from the group comprising zinc citrate, zinc lactate, zinc gluconate, zinc aspartate, zinc methionine, zinc oxide, zinc phosphate, zinc salicylate, and zinc chloride. Optionally, the zinc salt can be zinc citrate.

In some implementations, the composition can include at least 0.01 wt % of the zinc-containing compound. For example, the composition can include between 0.01 wt % and 25 wt % of the zinc-containing compound. For example, the composition can include between 0.01 wt % to 10 wt % of the zinc-containing compound. For example, the composition can include between 0.01 wt % and 1 wt % of the zinc-containing compound.

In some implementations, the composition can be configured for oral administration. For example, the composition can be edible.

In some implementations, the composition can be configured for topical application.

In some implementations, the composition can be configured for chewable delivery.

In some implementations, the composition further includes a gel base.

In some implementations, the composition can be an oral care product. For example, the oral care product is a mouthwash, a dentifrice, a paste, a spray, a dental chew, a gum, or a lozenge.

In some implementations, the composition can be a skin care product. For example, the skin care product can be a gel, a lotion, a cream, or a spray that is configured for topical application to a cutaneous membrane.

In some implementations, the composition can be a gastrointestinal care product. For example, the gastrointestinal care product can be a paste, a powder, a capsule, a gummy, a liquid, a suppository or a chewable substance.

In some implementations, the composition can be a paste, a powder, a gel, a liquid, or a chewable substance.

In another aspect, there is provided a method of preventing and/or treating inflammation in a subject comprising administration of a composition comprising A-type proanthocyanidin (PAC-A) and zinc. For example, the inflammation can be associated with an oral cavity, cutaneous membrane, or gastrointestinal tract. For example, the inflammation can be associated with at least one of gingivitis, periodontitis, muscle pain, acne, allergies, dermatitis, psoriasis, eczema, heat rash, photosensitivity, abrasion, stomatitis, lesions, and gastrointestinal inflammation. For example, the inflammation can be associated with dermatitis. For example, the inflammation can be associated with periodontitis.

In some implementations, the composition can be administered orally. In other implementations, the composition can be administered topically.

In some implementations, the method can include multiple exposures to the composition.

The subject can be a mammal. For example, the subject can be a human. For example, the subject can be an animal. For example, the subject can be a dog, a cat, or a horse.

In accordance with another aspect, there is provided an oral care product for reducing and/or preventing inflammation associated with an oral cavity in a subject, the oral care product comprising A-type proanthocyanidin (PAC-A) and zinc.

In some implementations, the inflammation is associated with periodontic diseases or stomatitis.

In some implementations, the oral care product comprises between 0.001 wt % and 10 wt % of PAC-A, optionally between about 0.0035% and about 0.42% PAC-A.

In some implementations, the PAC-A is provided as a PAC-A containing extract.

In some implementations, the oral care product comprises between 0.05 wt % and 15 wt % of the PAC-A containing extract, optionally between about 1 wt % and about 3 wt % PAC-A containing extract. In some implementations, the oral care product comprises between about 0.01 wt % and about 25 wt % of a zinc-containing compound, optionally between about 0.1 wt % and about 10 wt % of the zinc-containing compound, further optionally between about 0.1 wt % and about 1 wt % of the zinc-containing compound. For example, the zinc-containing compound is a zinc salt, such as zinc citrate. The PAC-A containing extract can be provided as a cranberry extract. For example, the cranberry extract can be one of a dried cranberry powder, a soluble cranberry extract, and an insoluble cranberry extract.

In another aspect, there is provided a use of the composition as defined herein for preventing and/or treating inflammation.

In another aspect, there is provided a use of the composition as defined herein for the manufacture of a medicament for the treatment of inflammation.

In another aspect, there is provided a process for producing an oral gel product. The process includes providing a gel base; and admixing a zinc-containing compound and an A-type proanthocyanidin (PAC-A)-containing extract to the gel base to form the oral gel product. For example, the admixing can be performed to form the oral gel product having between 0.001 wt % and 10 wt % of PAC-A. For example, the admixing can be performed to form the oral gel product having between 0.01 wt % and 10 wt % of the zinc-containing compound.

In some implementations, the PAC-A containing extract is a PAC-A containing cranberry extract.

In some implementations, the zinc-containing compound is a zinc salt.

In some implementations, the process can include maintaining a temperature preventing degradation of the PAC-As during admixing of the PAC-A containing extract and the zinc-containing compound to the gel base.

In addition, the method/process described herein can include at least one additional feature/step that is derived from the definition of the composition comprising PAC-A and zinc as provided herein.

While the invention will be described in conjunction with example embodiments, it will be understood that it is not intended to limit the scope of the invention to such embodiments. On the contrary, it is intended to cover all alternatives, modifications and equivalents as may be included as defined by the present description. The objects, advantages and other features of the present invention will become more apparent and be better understood upon reading of the following non-restrictive description of the invention, given with reference to the accompanying drawings.

### BRIEF DESCRIPTION OF THE DRAWINGS

FIG. 1 is a bar graph demonstrating the interleukin-6 (IL-6) production (pg/ml) by human organotypic models under inflammatory stress (bacterial lipopolysaccharides (LPS) and polyinosinic: polycytidylic acid (Poly I:C)) when treated with the formulations in Table 1;

FIG. 2 is a bar graph demonstrating the interleukin-8 (IL-8) production (pg/ml) by human organotypic models under inflammatory stress (LPS/Poly I:C) when treated with the formulations in Table 1;

FIG. 3 is a bar graph demonstrating the average expression levels (fold increase) of genes encoding IL-6 in human organotypic models under inflammatory stress (LPS/Poly I:C) when treated with the formulations in Table 1;

FIG. 4 is a bar graph demonstrating the average expression levels (fold increase) of genes encoding IL-8 in human organotypic models under inflammatory stress (LPS/Poly I:C) when treated with the formulations in Table 1;

FIG. 5 is a bar graph demonstrating the IL-6 production (pg/ml) by human organotypic models under inflammatory stress (LPS/Poly I: C) when treated with varying concentration levels of PAC-A containing extract (0%, 0.1%, 1%, 3%, 6%, and HBSS) being a PAC-A concentrated cranberry powder;

FIG. 6 is a bar graph demonstrating the IL-6 production and IL-8 production (pg/ml) by human organotypic models under inflammatory stress (LPS/Poly I:C) when treated with the formulations 2A, 2C, 2G and 2H as identified in Table 2;

FIG. 7 is a bar graph demonstrating the IL-8 production (pg/ml) by human organotypic models under inflammatory stress (LPS/Poly I:C) when treated with the formulations in Table 2;

FIG. 8 is a bar graph demonstrating the average expression levels (fold increase) of genes encoding IL-6 in human organotypic models under inflammatory stress (LPS/Poly I:C) when treated with the formulations in Table 2;

FIG. 9 is a bar graph demonstrating the average expression levels (fold increase) of genes encoding IL-8 in human organotypic models under inflammatory stress (LPS/Poly I:C) when treated with the formulations in Table 2;

FIG. 10 is a bar graph demonstrating the IL-6 production (pg/ml) by human organotypic models (gingival model) under inflammatory stress (LPS/Poly I:C) when treated with the formulations of Table 3;

FIG. 11 is a bar graph demonstrating the IL-6 production (pg/ml) by human organotypic models (gingival model) under inflammatory stress (LPS/Poly I:C) when treated with the formulations in Table 4; and

FIG. 12 is a bar graph demonstrating the IL-6 production (pg/ml) by human organotypic models (gingival model) under inflammatory stress (LPS/Poly I:C) when treated with the formulations in Table 5.

FIG. 13 is a bar graph showing antioxidant values (measured as antioxidation capacities TEAC) of different tested gels containing various active ingredients.

FIG. 14 is a bar graph showing the IL-6 production (pg/ml) by a skin model under inflammatory stress, when treated with cream formulations comprising at least one of PAC-rich cranberry 1 wt % and Zinc 0.2 wt %, having undergone a heat treatment at various temperatures for 30 minutes.

FIG. 15 is a bar graph showing the IL-6 production (pg/ml) by human organotypic models (gingival model) when treated with gel formulations including at least one Copper and PAC-rich cranberry.

FIG. 16 is a bar graph showing the IL-6 production (pg/ml) by human organotypic models (gingival model) when treated with gel formulations made with soluble and insoluble fractions of the cranberry, with variations in the sources of zinc, the zinc concentration and the temperature at which the gel is exposed when prepared.

### DETAILED DESCRIPTION

Examples, variants and preferred embodiments of the invention are described hereinbelow. In one aspect, there is provided a composition for treating and/or preventing inflammation in a subject comprising a synergistic combination of A-type proanthocyanidin (PAC-A) and zinc ions. More particularly, it has been surprisingly found, as demonstrated herein, that zinc ions act as a potentiator for the anti-inflammatory capacity of PAC-A and that a combination of zinc and PAC-A containing compounds/extracts can treat and/or prevent inflammation in a subject.

In some implementations, this composition can be used as a dental product for the treatment and/or prevention of inflammation associated with an oral cavity in a subject, such as an animal.

In the context of this description, the term “treatment” of inflammation can include reducing inflammation in the subject.

### A-Type Proanthocyanidin

Proanthocyanidins (PACs) are a class of polyphenols produced by natural sources, such as cranberries and blueberries and other members of the Ericaceae family, as well as other plants, such as aronia (chokeberries), cinnamon and cinnamon bark, grapes and grapeseed oil, strawberries, avocados, green tea, haksap berries, etc. PACs have a high antioxidant capacity, as well as anti-inflammatory and anti-bacterial properties. PACs can bind to bacteria to prevent them from latching on a mucous membrane, thus preventing growth of a biofilm or attachment of a colony in a subject. In this context, the term “subject” refers to a living vertebrate organism, such as a mammal, including, without limitation, humans, domestic animals, such as dogs or cats, farm, or livestock animals, such as horses, zoo animals, etc.

A-type proanthocyanidin (PAC-A) is a subclass of PACs with anti-bacterial and anti-inflammatory properties that can be found in natural sources, such as from Vaccinium oxycoccos and Vaccinium macrocarpon (cranberries). For example, PAC-As can be included in PAC-A containing extracts derived from PAC-A containing natural sources. In another example, PAC-As can be synthesized, such that the PAC-A containing extract can be prepared having a tailored concentration of synthesized PAC-As. When the PAC-A containing natural sources include cranberries, the PAC-A containing extract can be referred to as a PAC-A containing cranberry extract. The PAC-A containing extract as encompassed herein can include a soluble PAC-A containing extract, an insoluble PAC-A containing extract, or a combination thereof. Soluble extracts can be useful for formulations in a gel or dissolved in a liquid formulation for topical application, such as in a spray, or for oral ingestion. For example, the soluble portion of a dried cranberry powder (soluble extract) can be composed of shorter chain PAC-A polymers than the insoluble portion of a dried cranberry powder (insoluble extract). In another example, the PAC-A containing extract can be dried cranberry powder (including soluble and insoluble portions), that is obtained from a whole PAC-A containing natural source, i.e. cranberries. Pure dried cranberry powder can contain up to about 7 wt % PAC-A. For example, the PAC-A containing extract which derives from a natural source can have a PAC-A concentration ranging from about 1 wt % to about 15 wt % with respect to a total weight of the PAC-A containing extract. For example, the dried cranberry powder can have a PAC-A concentration of at least 5 wt %, at least 6 wt % or at least 7 wt %. However, it should be understood that a PAC-A containing extract can be prepared or synthesized according to any known techniques to increase the PAC-A concentration. It should be noted that the present techniques are not limited to a specific PAC-A concentration in the PAC-A containing extract. The synergistic effect toward treatment of inflammation can be observed for a composition comprising zinc and at least 0.001 wt % of PAC-A, at least 0.01% of PAC-A, at least 1% of PAC-A, or at least 10% of PAC-A with respect to a total weight of the composition. For example, the composition can include between about 0.001 wt % and about 10 wt % of PAC-A with respect to a total weight of the composition.

In the context of this description, the term “extract” or “PAC-A containing extract” is intended to include PAC-A containing compounds that result from a physical and/or chemical treatment of a PAC-A containing natural source to recover a PAC-A content thereof in the resulting extract. The physical and/or chemical treatment can be tailored to obtain a higher PAC-A concentration in the resulting extract (such as soluble extract, insoluble extract, or dried cranberries). A soluble extract includes a solution of solubilized cranberry pomace and can be prepared by dehydration of a filtrate. An insoluble extract includes the non-solubilized fraction of the previously mentioned soluble cranberry pomace and can be prepared by dehydration. Dried cranberries can be further grinded to produce a pure dried cranberry powder that can be used as the PAC-A containing extract.

PAC polymers have a reactive binding site on their end units that can accommodate large ions. For example, the reactive site of PAC-A polymer can accommodate zinc ions, thereby forming a PAC-A/zinc complex. Specifically, PACs are polymers composed of two flavanol subunits, namely catechin and epicatechin, linked by two different types of bonds. The bond in B-type proanthocyanidins (PAC-Bs) is a mono-covalent link between carbons C4 and C8 (b-type bond), while the bond in A-type proanthocyanidin (PAC-As) includes a second interflavanic bond between the carbon C2 and the oxygen of carbon C7 (a-type linkage). The a-type linkage between catechin and epicatechin in PAC-A enables the binding of a zinc ion (Zn²⁺) to an end unit of two PAC-A polymers, thus binding them together into the PAC-A/zinc complex.

It should be noted that commercially available cranberry extracts/products, such as cranberry powder, may not include PAC-As depending on the method of production of the cranberry extract/product. In addition, the PAC-A concentration in a cranberry extract may vary from one type of extract to another. It should further be noted that subjecting the cranberry source or extract to a heat treatment at a temperature can degrade the PAC-As and render the cranberry extract inefficient for the purpose of the present invention targeting a synergistic effect between zinc ions and PAC-As. Experimental results are provided to show that exposure to an excessive temperature can hinder the anti-inflammatory function of the synergistic combination of PAC-A and zinc in the composition. The composition encompassed herein is prepared according to techniques that do not expose the PAC-A containing extract to any heat that would cause a reduction or suppression of the synergistic effect toward inflammation when combined with a zinc-containing component.

### Zinc

Zinc ions are known to have anti-bacterial properties and can be used in oral hygiene products, such as mouth rinses or sprays and dentifrices to control dental plaque, inhibit calculus formation and reduce halitosis.

However, it is shown herein that zinc ions provided by a zinc-containing compound, also referred to as a zinc-based compound, in combination with a PAC-A containing extract, can act as a potentiator for the anti-inflammatory capacity of PAC-A. The zinc-containing compound as encompassed herein can be or include, without limitation, zinc salts or a combination thereof. Zinc salts include zinc citrate, zinc lactate, zinc gluconate, zinc aspartate, zinc methionine, zinc oxide, zinc phosphate, zinc salicylate, zinc chloride, and any combinations thereof.

It should be noted that the concentration of the zinc-containing compound in the composition may be limited by the viability of the subject zone to be treated.

### Potentiating the Effect of PAC-A

The effect of PAC-A containing extracts in reduction/suppression of in vitro expression of inflammation-associated interleukin-6 (IL-6) and interleukin-8 (IL-8) genes was found to be favourably altered in the presence of zinc ions. As seen in the example section, a composition comprising 3 wt % PAC-A containing cranberry extract (dried cranberry powder), without the use of a potentiator, was found to be optimal to reduce the expression of the interleukin-6 and interleukin-8 genes and production of IL-6 and IL-8, thus optimal in reducing inflammation in a subject's tissue. When the PAC-A containing cranberry extract was combined with zinc citrate, the concentration of the PAC-A containing cranberry extract could be reduced while still exhibiting substantially similar suppression of IL-6 and IL-8 gene expression and IL-6 and IL-8 production, thus showing that zinc ions can be a potentiator for the anti-inflammatory properties of PAC-A.

Thus, compositions that combine PAC-A with zinc can utilize a lesser quantity of PAC-A that would normally be necessary to reduce inflammation in the tissues of a subject. As seen in the example section, the use of zinc ions as a potentiator can reduce the total concentration of PAC-A containing extracts required in an effective dosage to at least 1 wt % PAC-A containing extract in the composition, while maintaining similar levels of IL-6 and IL-8 gene expression and IL-6 and IL-8 production as the optimal level of 3 wt % PAC-A, when used without a potentiator. In some implementations, the use of zinc as a potentiator can reduce the concentration of the PAC-A containing extract in the composition to at least about 0.05 wt %. As seen in the example section, the potentiating effect of zinc was demonstrated in extracts containing PAC-A, yet was not shown in extracts containing B-type proanthocyanidins (PAC-B), such as blueberry extracts. The a-type linkage of PAC-A polymers creates a reactive site that can stably bind to a zinc ion with a second PAC-A polymer, whereas the single bond b-type link between epicatechin and catechin in PAC-B polymers does not.

In another example, a composition containing at least 1 wt % of PAC-A containing extracts and at least 0.01 wt % of zinc-containing compound has been shown to reduce expression of the interleukin-6 and interleukin-8 genes and production of IL-6 and IL-8, thus reduce inflammation. Inclusion of at least 0.01 wt % of zinc-containing compound with PAC-A containing extracts has been shown to increase the anti-inflammation properties of the PAC-A. Using zinc ions as a potentiator of the anti-inflammatory properties of PAC-A reduces the total concentration of anti-inflammatory PAC-A compounds necessary in an effective dose of the composition. As most PAC-A containing ingredients or extracts are brightly coloured (such as cranberries), a reduction in the amount of PAC-A containing ingredients facilitates the creation of a colourless or easily dyed formulation. Furthermore, using zinc ions as a potentiator of the anti-inflammatory capacity of PAC-A allows for an anti-inflammatory composition with higher potency than formulations without zinc ions.

In some embodiments, the composition can comprise between about 0.05 wt % and about 15 wt % PAC-A containing extract, between about 0.05 wt % and 6 wt % of PAC-A containing extract, or between about 1 wt % and 3 wt % of PAC-A containing extract, to achieve a PAC-A concentration of least 0.001 wt %, at least 0.01 wt %, at least 0.1 wt %, at least 1 wt %, or at least 10 wt % in the composition (in accordance with the PAC-A concentration of the PAC-A containing extract. For example, the composition can include between about 0.001% and about 10% of PAC-A with respect to a total weight of the composition, optionally between about 0.0035% and about 0.42% of PAC-A. The PAC-A containing extract can be derived from a natural source, such as cranberries. The PAC-A containing extract can be soluble or insoluble. In some implementations, the PAC-A containing extract is a dried cranberry powder.

In some implementations, the composition comprises between about 0.01 wt % and about 25 wt % of the zinc-containing compound, for example between about 0.01 wt % and about 10 wt % of the zinc-containing compound, optionally between 0.01 wt % and 1 wt % of the zinc-containing compound. For example, the composition can comprise at most 3 wt %, at most 2 wt %, or at most 1 wt % of the PAC-A containing extract, and at most 0.6 wt %, at most 0.2 wt % or at least 0.01 wt % of a zinc salt, such as zinc citrate.

### Other Effects

It should be noted that the active ingredients described herein, namely PAC-A and zinc ions, can provide more than an anti-inflammatory function/effect. It is not intended to limit the application of the composition described herein to treatment of the inflammation, and the composition could be further used for an antioxidation function, for the reduction/prevention of bacterial growth or for supporting immune response.

### Delivery Methods

The composition comprising PAC-A and zinc can be used to treat a variety of ailments or conditions caused by inflammation of the oral cavity (including periodontal and stomatitis diseases, such as periodontitis or gingivitis), inflammation of the skin (including acne, hives (allergies), dermatitis, psoriasis, eczema, heat rash, photosensitivity, abrasions, and lesions), and/or gastrointestinal inflammation (including pharyngitis or inflammatory bowel disease). As such, delivery methods for the composition can for example include topical application on the affected tissue, oral administration, or chewable delivery. In this context, the term “topical application” means a formulation that is configured for application to a body surface, including the oral cavity (mouth, gums, etc. . . . ) and the cutaneous membrane (skin) for delivery of the active substance through dermal, sublingual, buccal, or oral mucosal absorption. The term “oral administration” means a formulation that is configured for delivery of the active substance to the gastrointestinal tract for absorption. The term “chewable delivery” means a formulation that is configured to be chewable for topical delivery to the oral cavity, such as gum or a dental chew. The compositions described herein can be delivered to the tissue (before or when being affected/inflamed) via a variety of delivery methods, including, without limitation, a gel, dentifrice, dental chew, lotion, spray, cream, gum, drink, tablets, lozenges, capsules, syrups, capsules, water-soluble or oil suspensions, etc.

In some embodiments, especially compositions configured for oral administration, chewable delivery, or topical application to the oral cavity, the composition can be comprised entirely of edible ingredients, such as dried cranberry powder and zinc citrate.

In some embodiments, the composition is configured for topical application to the oral cavity or oral mucosa, including the gums and buccal cavity. For example, with periodontitis, inflammation of the gums is the main cause of the loss of bone density. Matrix metalloproteinases produced by the inflammation cause the degradation of the alveolar bone by chelating calcium. Reducing inflammation in the subject's gums therefore reduces the potential loss of alveolar bone. Compositions configured for topical application to the oral cavity or oral mucosa can include a gel, dentifrice, oral spray, mouthwash, lozenge, etc.

In some embodiments, the composition is configured for topical application to the cutaneous membrane (skin). For example, inflammatory acne can result from clogged skin pores. Treatment of the inflammation of the skin can reduce the swelling and redness of the clogged pores. Muscle pain can also be caused by inflammation of the muscle tissues; reducing the inflammation in the affected tissue can reduce the associated muscle pain. Compositions configured for topical application to the cutaneous membrane can include a gel, a lotion, a cream, or a spray. In other embodiments, the composition can be used in cosmetic products, such as scar or skin rejuvenation cream, makeup primer, concealer, etc.

In some embodiments, the composition is configured for oral administration. For example, pharyngitis is caused by inflammation of the pharynx (i.e., the portion of the throat between the mouth/nasal cavity and the oesophagus/trachea), which can cause pain and swelling of the throat. Reducing inflammation in the pharynx can reduce the associated symptoms. Oral administration for delivery of the composition to the gastrointestinal tract can include compositions in the form of a gel, a spray, a drink, tablets, lozenges, capsules, water or oil suspensions, syrups, etc.

In some embodiments, the composition is configured for chewable delivery, such as for delivery to the oral mucosa to treat periodontal diseases. Chewable deliveries are especially useful for non-human subjects to maximize the time of exposure to the oral mucosa. For example, the composition can be in the form of a dental chew for administration to the oral mucosa of domestic animals such as dogs or cats. In other embodiments, such as for human subjects, the composition can be in the form of chewing gum for prolonged exposure to the oral mucosa.

For example, the composition can be an oral care product comprising at most 3 wt %, at most 2 wt %, or at most 1 wt % of the PAC-A containing extract, such as the dried cranberry powder, and at most 0.6 wt %, at most 0.2 wt % or at least 0.01 wt % of a zinc salt, such as zinc citrate. In another example, the composition can be a skin care product comprising at most 3 wt %, at most 2 wt %, or at most 1 wt % of the PAC-A containing extract, such as the dried cranberry powder, and at most 0.6 wt %, at most 0.2 wt % or at least 0.01 wt % of a zinc salt, such as zinc citrate.

In some implementations, other compounds that are beneficial to the health of the subject or to improve palatableness of the composition can be included as compounds. For example, natural extracts containing PAC-B antioxidants, such as extracts isolated from blueberries, aronia (chokeberry), haskap berries, green tea, strawberries, grapes, and grapeseed oil, grapefruit, lemon and cherries can be included in the composition. In some implementations, compounds such as vitamin C, vitamin D, vitamin E, omega-3 fatty acids, omega-6 fatty acids, sodium hexametaphosphate, pyrophosphate, dicalcium phosphate, citric acid, lactoperoxidase, glucose oxidase, lysozyme, and/or proteases can be included in the composition. In some implementations, agents such as humectants, thickeners, wax, flour, starch, emulsifiers, and/or oil can be used as part of the delivery system of the composition.

### Example Implementations

Provided below is an example of a dental gel product:

| Component | Wt. % |
|---|---|
| PAC-A containing | 3.0% |
| cranberry extract | |
| Zinc citrate | 0.2% |
| Water | 70% |
| Humectant | 9% |
| Thickener | 6% |
| Other | 11.8% |

Provided below is an example of a dental chew composition:

| Component | Wt. % |
|---|---|
| PAC-A containing | 3.0% |
| cranberry extract | |
| Zinc citrate | 0.1% |
| Flour | 45% |
| Water | 25% |
| Starch | 20% |
| Flavor | 1.9% |
| Other | 5% |

Provided below is an example of a health drink composition:

| Component | Wt. % |
|---|---|
| PAC-A containing | 3.0% |
| cranberry extract | |
| Zinc citrate | 0.2% |
| Water | 90% |
| Other | 6.8% |

Provided below is an example of a lotion composition:

| Component | Wt. % |
|---|---|
| PAC-A containing | 1.0% |
| cranberry extract | |
| Zinc citrate | 0.2% |
| Water | 80% |
| Oil | 10% |
| Emulsifier | 4% |
| Humectant | 4% |
| Thickener | 0.8% |

Provided below is an example of a cream composition:

| Component | Wt. % |
|---|---|
| PAC-A containing | 3.0% |
| cranberry extract | |
| Zinc citrate | 0.2% |
| Water | 53% |
| Oil | 35% |
| Wax | 2% |
| Thickener | 0.5% |
| Other | 6.3% |

It should be noted that other products can be produced to provide the composition to a subject. For example, a capsule containing the composition as encompassed herein can be used to treat gastrointestinal inflammation. Optionally, the composition in the capsule can include at least 80 wt % of the PAC-A containing extract and 1 wt % of zinc citrate. For example, the composition in the capsule can include about 99 wt % of a dried cranberry powder and about 1 wt % zinc citrate.

### EXPERIMENTATION RESULTS

### Example 1-Effects of Compositions Containing PAC and Zinc on In Vitro Organotypic Models-detection of Pro-inflammatory Cytokines (IL-6 and IL-8)

An in vitro assay was performed to show the enhanced anti-inflammatory effect of PAC-A and zinc compositions on artificial human organotypic models. An EpiGingival™ three-dimensional organotypic model (GIN-112 from MaTtek™) was used to determine the inhibitory effect of the synergistic combination of PAC-A and zinc ions on pro-inflammatory cytokines production. The organotypic model is equivalent to a human oral mucosa formed by a multilayer of highly differentiated human keratinocytes. Briefly, human epithelial cells isolated from healthy volunteers were deposited on a surface of a porous membrane bathed in a culture medium containing growth factors. Cellular multilayers formed with an air-liquid interface with the apical (upper) portion of the model in contact with the environment (air) while the basolateral portion, which is supported by the porous membrane, is in contact with the culture medium. The models were incubated (37° C., 5% CO₂) overnight and then inoculated with an immunostimulant medium containing 10 μg/mL of polyinosinic: polycytidylic acid (poly I:C) and 2 μg/mL of bacterial lipopolysaccharides (LPS) to induce an inflammatory reaction. 40 μL of the tabled formulations were deposited on the surface of the organotypic models prior to a 24-hour incubation (37° C., 5% CO₂) period.

The organotypic models were rinsed with cold PBS to remove residual traces of the gel and stored in RNAlater™. In addition to the detection of pro-inflammatory cytokines (IL-6 and IL-8) in the culture medium, the analysis of the expression of genes encoding the pro-inflammatory cytokines was performed. The organotypic models were transferred to a Precellys™ type tube containing CK14 ceramic beads with 600 μL of cold RLT buffer (EZNA kit, Omega™). The samples were homogenized with a Precellys™ homogenizer at 6500 rpm for 30 seconds. The cellular lysates were then used to extract the RNA using the Omega™ EZNA kit according to the manufacturer's recommendations. The RNA was then eluted in 60 μL of elution buffer. 500 ng of RNA was transcribed into complementary DNA (cDNA) using the Qiagen™ retrotranscriptase (RT2 First Strand) according to the manufacturer's instructions. Amplification was carrier out to a volume of 20 μL using a thermocycler (Mx3000p, Stratagene™) using primers from Qiagen™ The sample was denatured for an initial cycle of 10 minutes at 95° C. and then 40 cycles alternating denaturation of 15 seconds at 95° C. and pairing of 60° C. were performed. Fluorescence was measured at the end of each cycle. The expression of housekeeping genes glyceraldehyde-3-phosphate dehydrogenase (GAPDH) and β-glucuronidase (GUSB) were also monitored. The Delta-Delta Ct method (ΔΔCt) was used to quantify the expression of each of the genes. The sample serving as the reference base for the calculations was the RNA extracted from the cells only in contact with the complete medium, Hank's Balanced Salt Solution (HBSS).

The following formulations were tested:

TABLE 1

| Formulation | Active Ingredients |
|---|---|
| 1A | HBSS control |
| 1B | 3% PAC-A containing cranberry extract |
| 1C | 3% PAC-A containing cranberry extract |
| 1D | 3% blueberry extract |
| 1E | 0.2% zinc citrate |

TABLE 2

| Formulation | Active Ingredients |
|---|---|
| 2A | Gel base |
| 2B | 3% PAC-A containing cranberry extract |
| 2C | 3% aronia extract (chokeberry) |
| 2D | 0.5% hydrocortisone |

TABLE 3

| Formulation | Active Ingredients |
|---|---|
| 3A | Gel base |
| 3B | 3% PAC-A containing cranberry extract |
| 3C | 1% PAC-A containing cranberry extract |
| 3D | 0.2% zinc citrate |
| 3E | 1% PAC-A containing cranberry extract + 0.2% zinc citrate |

TABLE 4

| Formulation | Active Ingredients |
|---|---|
| 4A | Gel base |
| 4B | 3% PAC-A containing cranberry extract |
| 4C | Soluble PAC formulation |
| 4D | Insoluble PAC formulation |
| 4E | 1 year old 3% PAC-A containing cranberry extract |
| 4F | 3% PAC-A containing cranberry extract + 0.6% zinc |
| | citrate |
| 4G | 1% PAC-A containing cranberry extract + 0.05% zinc |
| | citrate |
| 4H | 1% PAC-A containing cranberry extract + 0.0125% |
| | zinc citrate |

TABLE 5

| Formulation | Active Ingredients |
|---|---|
| 5A | Gel base |
| 5B | 3% PAC-A containing cranberry extract |
| 5C | 1% PAC-A containing cranberry extract |
| 5D | 1% blueberry extract (powder) |
| 5E | 0.2% zinc citrate |
| 5F | 1% PAC-A containing cranberry extract + 0.2% zinc citrate |
| 5G | 1% blueberry extract + 0.2% zinc citrate |

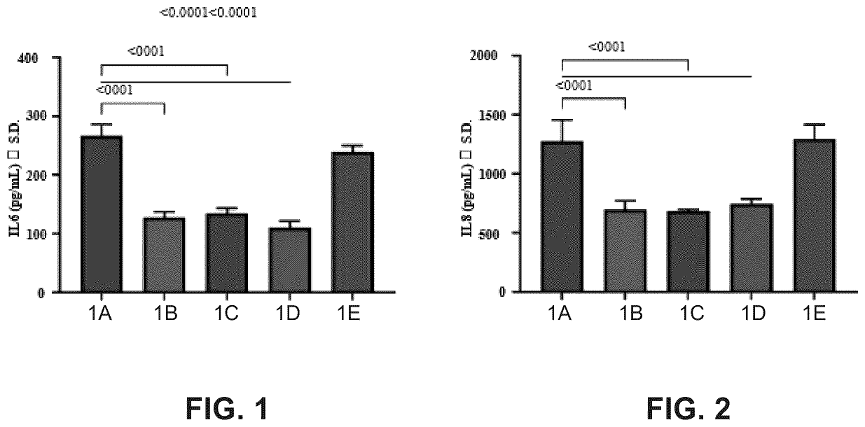

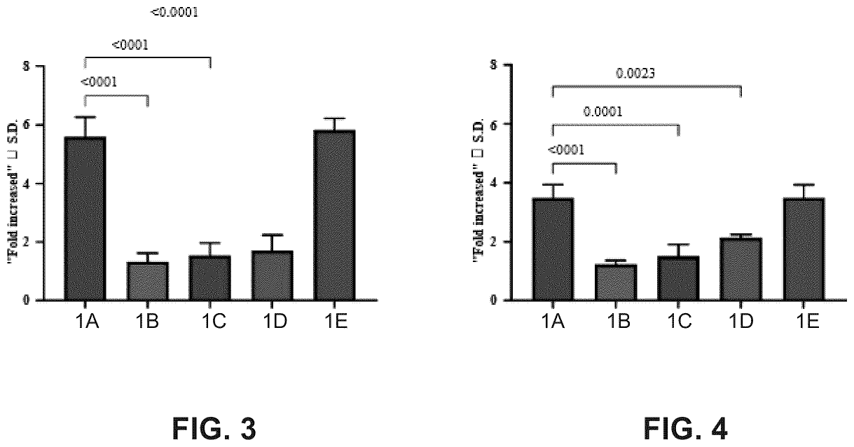

Referring now to FIGS. 1 to 4, the results showed that formulations with 3% PAC-A containing cranberry extract (formulations 1B and 1C) and 3% PAC-B containing blueberry extract (formulation 1D) significantly reduced the expression of IL-6 and IL-8 genes and the production of IL-6 and IL-8 over the control formulation containing HBSS (formulation 1A). No significant differences were found between the formulations containing only PAC-A containing cranberry extract or only PAC-B containing blueberry extract. The formulation with 0.2% zinc citrate (formulation 1E) did not yield any significant difference in the expression of the IL-6 or IL-8 genes or the production of IL-6 or IL-8.

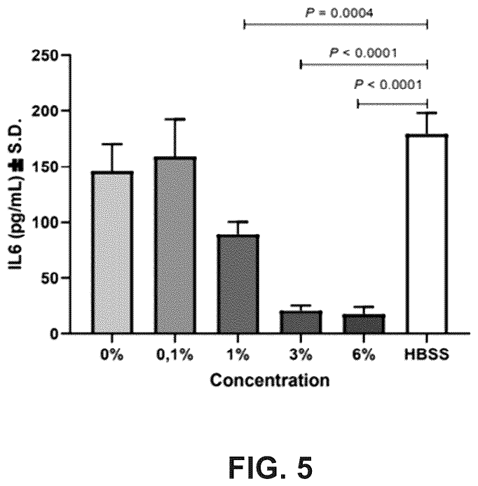

Referring now to FIG. 5, the bar graph shows IL-6 production (pg/ml) by human organotypic models under inflammatory stress (LPS/Poly I:C) when treated with 0%, 0.1%, 1%, 3%, and 6% PAC-A containing cranberry extract with a formulation containing HBSS as a control. The lowest optimal concentration of PAC-A containing extracts, without the use of a potentiator, for reducing the production of IL-6 was found to be at least 3%. There was a significant decrease in IL-6 production between the formulation containing 1% PAC-A containing extracts and the formulation containing 3% PAC-A containing extracts, and the production of IL-6 was not significantly reduced when a formulation with an increased PAC-A concentration of 6% was applied. As such, a concentration of 3% PAC-A containing extracts was used as a positive control in subsequent experiments.

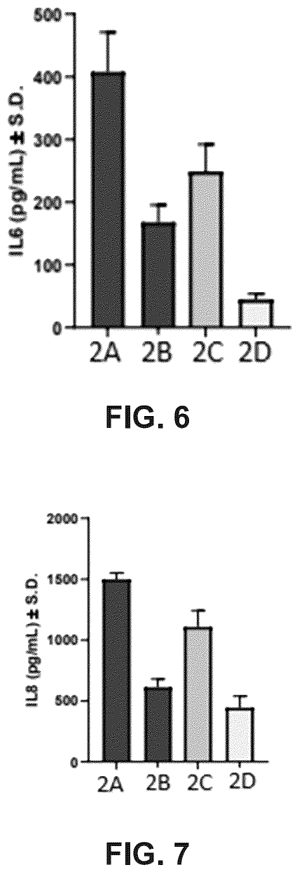

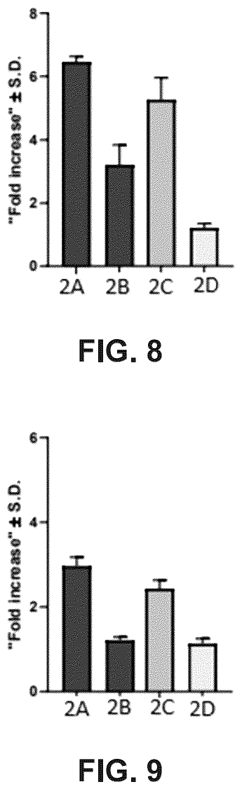

Referring now to FIGS. 6 to 9, the results confirmed that IL-6 and IL-8 gene expression and the cytokine concentration levels are significantly reduced in organotypic models treated with formulations with 3% PAC-A containing cranberry extracts (formulations 2A) for 24 hours. A comparison of the IL-6 production in organotypic models treated with formulations 2B and 2D show that the anti-inflammatory function of the cranberry PAC-A composition (2B) is comparable to that of the 0.5% hydrocortisone composition

Experimentation has thus shown that the PAC-A containing cranberry extract produces the majority of the anti-inflammatory effect as the reduction in pro-inflammation cytokines production is more pronounced when the concentration of cranberry extract is higher.

A formulation with 3% aronia berry (chokeberry) extract (formulation 2C) was also tested on the organotypic models. Referring to FIGS. 6 and 7, results showed that the formulation with PAC containing aronia berry extract (formulation 2C) resulted in a decreased production of IL-6 and IL-8 over the gel base control; however, the anti-inflammatory properties of aronia berry were not as pronounced as that of cranberry extract. Referring to FIGS. 5, 17 and 18, as the trend observed for IL-6 and IL-8 gene expression, and IL-6, IL-8, TNF-α and NF-KB production appeared similar, the further testing that was performed was based on evaluation of the IL-6 production only.

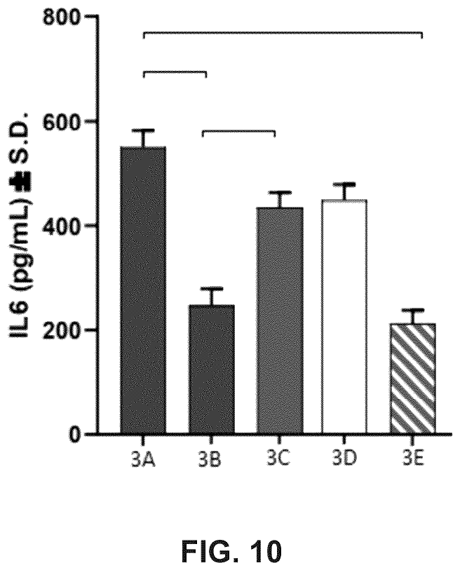

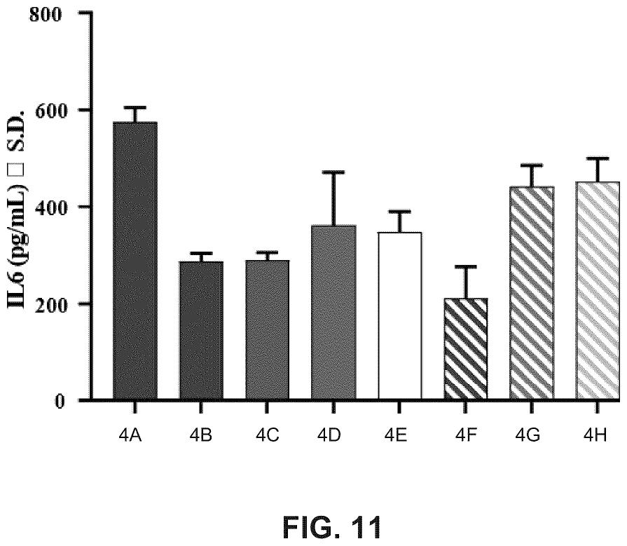

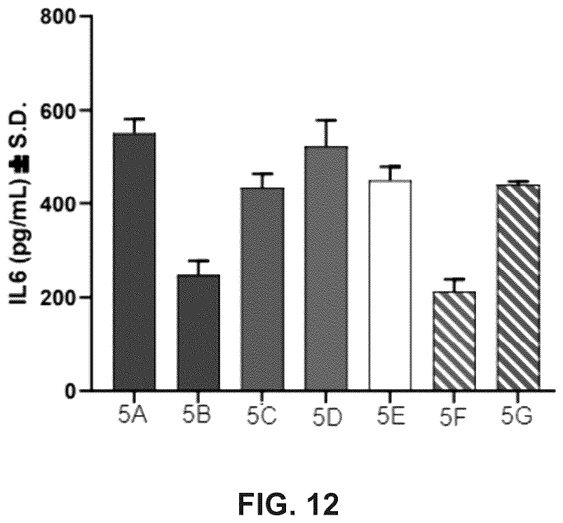

Referring now to FIG. 10, similar to the results shown in FIGS. 5 and 6, the formulations with 3 wt % PAC-A containing cranberry extract (formulation 3B) showed significantly less pro-inflammation cytokine production than the formulations comprising only 1 wt % PAC-A containing cranberry extract (formulation 3C). However, when 0.2 wt % zinc citrate was combined with 1 wt % PAC-A containing cranberry extract (formulation 3E), the production of IL-6 cytokines was significantly reduced to a comparable production level as the formulation containing 3 wt % PAC-A containing cranberry extract. As the formulation containing only 0.2 wt % zinc citrate (formulation 1D) did not significantly reduce the production of IL-6 cytokines over the HBSS control (FIG. 1), the results indicate that the zinc acts as a potentiator for the anti-inflammatory capacity of PAC-A. A comparison of the resulting IL-6 production in organotypic models treated with the formulation containing 1% PAC-A containing cranberry extract and 0.2% zinc citrate (formulation 3E) and the formulation containing 1% PAC-B containing blueberry extract and 0.2% zinc citrate (formulation 5G, FIG. 12) surprising shows that the potentiating effect of zinc ions is present with the PAC-A containing cranberry extract and absent with the PAC-B containing blueberry extract.

Further experimentation was conducted with various levels of zinc citrate, including 0.6 wt %, 0.05 wt %, and 0.0125 wt %, as shown in FIG. 11. IL-6 production was significantly reduced in the formulation containing 3 wt % PAC-A containing cranberry extract and 0.6 wt % zinc citrate (formulation 4F). Formulations 4G and 4H, containing 0.05 wt % zinc citrate or less did not exhibit the potentiating effect shown in formulations containing at least 0.2 wt % zinc citrate (formulations 3E and 4F).

Formulations containing soluble PAC-A containing cranberry extracts and insoluble PAC-A containing cranberry extracts were also applied to the organotypic models. There was no significant difference between the IL-6 cytokine production with models treated with 3 wt % PAC-A containing cranberry extract (formulation 4B) and the models treated with either soluble PAC-A containing cranberry extracts or insoluble PAC-A containing cranberry extracts (formulations 4C and 4D, respectively).

### Example 2-Effects of Compositions Containing PAC and Zinc Using H-ORAC Assays-study of Antioxidant Capacity

The antioxidant capacity of gels including separate active ingredients and a few mixtures of ingredients was assessed by using the Trolox Equivalent Antioxidant Capacity (TEAC) and the Hydrophilic-Oxygen Radical Absorbance Capacity (H-ORAC) potential. Table 6 provides a summary of the TEAC results for neutral gels containing separate ingredients (cranberry, blueberry, zinc citrate), mixtures of the ingredients and a reference neutral gel (gel 4) being assessed at 595 TEAC.

TABLE 6

| | | | % Efficiency |
|---|---|---|---|
| | | Results | compared to |
| Formulations | Active ingredient(s) | (TEAC) | neutral gel |
| Gel 1 | Cranberry 3% | 1812 | 204.54% |
| Gel 2 | Blueberry 3% | 1576 | 164.87% |
| Gel 3 | Zinc citrate 0.2% | 557 | −6.39% |
| Gel 4 | Neutral gel | 595 | 0.0% |
| Gel 5 | Cranberry 1% | 1018 | 71.09% |
| Gel 6 | Blueberry 1% | 1156 | 94.29% |
| Gel 7 | Cranberry 1%, Zinc | 1997 | 235.63% |
| | citrate 0.2% | | |
| Gel 8 | Cranberry 3.0%, Zinc | 2862 | 381.01% |
| | citrate 0.2%, | | |
| | Blueberry 3.0% | | |
| Gel 9 | Blueberry 1.0%, Zinc | 1165 | 95.80% |
| | citrate 0.2% | | |

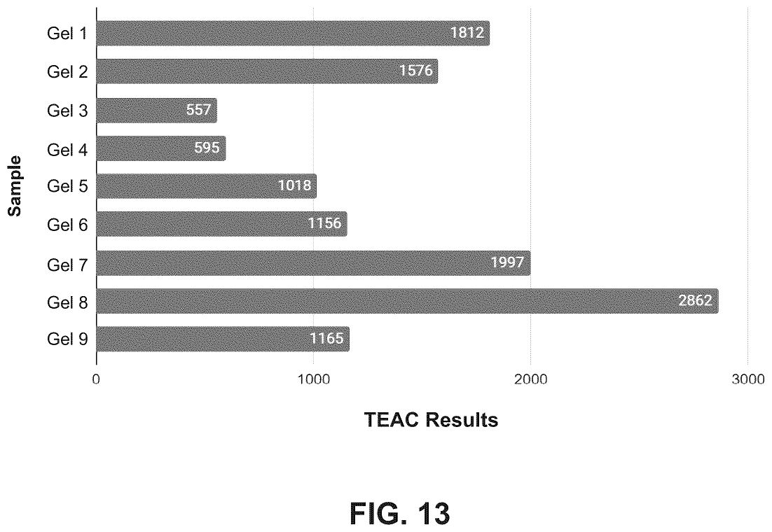

Referring to Table 6 and FIG. 13, it was found that the gel made with 0.2 wt % zinc citrate had a TEAC value just under the value measured of the neutral gel. A gel made with 1 wt % of cranberry powder had a measured TEAC value of 1018. When 0.2 wt % of zinc citrate was added to 1 wt % cranberry powder, the antioxidant capacity value was measured to be 1997 TEAC, which is 185 TEAC more than when 3 wt % cranberry powder alone was used, which is used as a positive control. This supports the idea that using zinc or a zinc-based compound, such as zinc citrate, and a PAC-rich cranberry powder increases the antioxidant capacity of the system.

A synergistic effect was not observed when a PAC-rich blueberry powder was used. Thus, the synergistic effect regarding the increase of the antioxidant capacity/potential is observed when using cranberry proanthocyanidins, namely PAC-A.

### Example 3-Effects of Compositions Containing PAC and Zinc on a Skin Model-study of Anti-inflammatory Effect by Detection of Pro-inflammatory Cytokines (IL-6)

Another series of experiments was performed to measure the anti-inflammatory effect of PAC-rich cranberry powder and zinc citrate in a different medium applied on a different type of human tissue. In this case, active ingredients were provided in an oil-based commercially available skin cream and applied on a skin model (MaTtek EpiDerm™). Two hypotheses were tested in this series of experiments. The first hypothesis was that the cranberry-zinc synergy is not dependent on the medium nor the tissue model. The second hypothesis is that the synergy is temperature dependent.

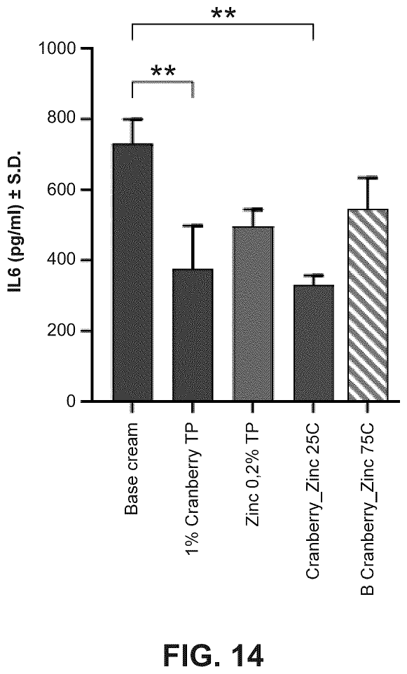

Referring to FIG. 14, the commercially available skin cream that was enriched with a PAC-rich cranberry powder (1 wt %) was found to have a significantly different anti-inflammatory effect from a neutral cream that was used as reference. The cream that was enriched with only zinc citrate (0.2 wt %) did not yield any significant difference in anti-inflammatory effect compared to the neutral cream. When a combination of 1.0 wt % of PAC-rich cranberry powder and 0.2 wt % of zinc citrate was added to the cream (without heat treatment), a significantly different and higher anti-inflammatory effect was observed. This cream was found to be the most effective, when compared to the other creams. This suggests that the synergy can be observed in a different medium than a gel. It also suggests that the synergy works on different types of human tissues (e.g. gingival model and skin model), and when used in a different medium.

In the second half of the experiment, the same commercially available cream was supplemented with 1.0 wt % of PAC-containing cranberry powder and 0.2 wt % of zinc citrate but heated to a temperature of 75° C. for a duration of 30 minutes. Still referring to FIG. 14, the cream sample did not demonstrate any synergistic anti-inflammatory response, thereby showing that temperature can be detrimental to the PAC-A-zinc synergy.

### Example 4-Effects of Compositions Containing PAC and Copper on a Gingival Model-study of Anti-inflammatory Effect by Detection of Pro-inflammatory Cytokines (IL-6)

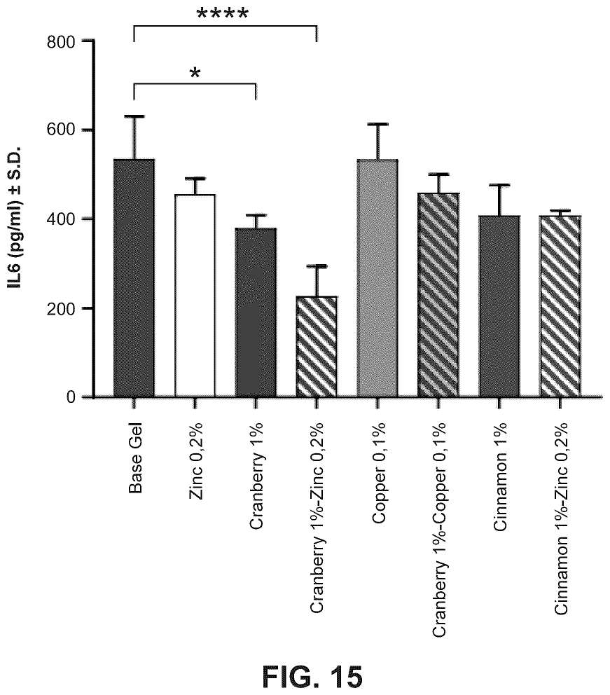

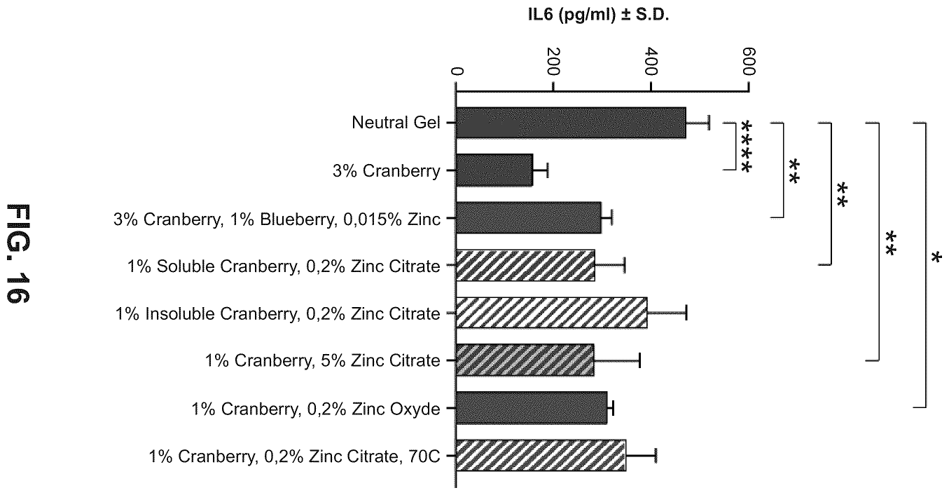

Another series of experiments was performed to evaluate the effect of a different metallic ion than zinc. These trials were done using gel as a medium and a gingival tissue model (MaTtek EpiGingival™) as described in Example 1. Copper (2+) citrate was used to substitute the zinc citrate. The concentration of copper used (0.1046 wt %) was used to have the same concentration of copper and zinc ions. Cu²⁺ was chosen as a substitute as it is very close in atomic radius, in atomic weight and in electronic distribution to Zn²⁺. Referring to FIG. 15, when copper was used alone in a gel, there was no significant difference between this gel and the neutral gel (gel base). When the gel was made including cranberry and copper citrate, the IL-6 production was not significantly different from the one of the gel made with only copper or the neutral gel. This data supports the hypothesis that only Zn²⁺ ions have a potentiator effect with cranberry.

### Example 5-Effects of Compositions Containing PAC and Zinc on a Gingival Model-study of Anti-inflammatory Effect by Detection of Pro-inflammatory Cytokines (IL-6)

Another series of experiments was performed on the gingival model (MaTtek EpiGingival™) as described in Example 1 to characterize the role of the soluble and non-soluble fractions of the cranberry in the observed synergy regarding reduction of inflammation. The separation of the soluble and the non-soluble fractions of the PAC-A containing cranberry extract was performed and two different gel samples were made using a concentration of 1 wt % in the soluble fraction and 1 wt % of the non-soluble fraction of the cranberry powder respectively, and a concentration of 0.2 wt % of zinc citrate. Referring to FIG. 16, the gel sample made with the soluble fraction showed an anti-inflammatory result significantly different from the neutral gel, whereas the gel sample made only with the insoluble fraction did not. Thus, the non-soluble fraction did not seem to give the same synergy as the soluble fraction. The gel made with only 1 wt % of the soluble fraction and 0.2 wt % zinc citrate did not achieve a synergy as strong as when the whole cranberry powder is used. In other experiments, a gel made with 1 wt % whole cranberry powder and 0.2 wt % zinc citrate would have the same effect as 3 wt % cranberry powder. In this case, the 3 wt % cranberry gel and the gels made with 1 wt % soluble and insoluble cranberry fractions along with 0.2 wt % zinc citrate are all significantly different from the neutral, suggesting that both fractions could demonstrate a synergistic effect.

The zinc upper limit of the zinc utilization has been further tested. A concentration of 5 wt % of zinc citrate has been for example used. The hypothesis is that Zn2+ions replenish the antioxidant potential of the PACs when it has been used. 5 wt % of zinc citrate corresponds to a maximal concentration of 1,745 wt % of Zn²⁺ ions in the gel. The maximal concentration to be used corresponds to an upper limit of zinc concentration over which a precipitate in the gel is formed and/or degradation of the tested in vitro tissue model is observed. Still referring to FIG. 16, the gel including 5 wt % zinc citrate and 1 wt % cranberry did not give any better results than the gel made with the soluble cranberry fraction. A viability assay was done on the gingival model after that addition of the maximal zinc concentration, and it was found that the viability had significantly decreased. This could have influenced the production of the interleukin IL-6 on the model. The very high concentration of zinc ions could probably explain the low synergy. Since only 1 wt % cranberry powder was used, PACs were the limiting reactant. This example series of experiments represents the only occurrence where the proanthocyanidins (PACs) were used as reactants with a limiting concentration. This means that zinc does not replenish the antioxidizing capacity of the PACs, like it was previously hypothesized. Otherwise, a great synergy would have been observed.

A different zinc salt has been used to understand if the citrate ions are necessary for the synergy to take place. A gel was made with 1 wt % of cranberry powder and 0.0875 wt % zinc oxide. This concentration represents an equivalent Zn²⁺ ions concentration as the usage of 0.2 wt % zinc citrate. This gel does show similar results to the soluble cranberry and zinc citrate gel. These results showed that zinc oxide can act as a potentiator to get an effect similar to a gel made with a higher concentration of PAC-A containing extract (see previously reported results).

The influence of heat on the system has been studied again. A gel made with 1.0 wt % cranberry and 0.2 wt % zinc citrate gel has been placed in a hot water bath at 70° C. for 30 minutes. This heating regiment has been used to try to denature a maximum of PACs in the cranberry. By changing the concentration of PACs, it has been hypothesized that the synergy should be less effective. The results of this trial could be compared to the soluble and the insoluble fractions done on the same model. When applied to the gingival model, the cooled gel did not show any result significantly different from the neutral gel. This means that the synergy can be decreased and possibly deactivated when the medium is heated. When the synergy is reduced by heating, it is most likely not because the PACs are completely degraded.

In the above description, the term “about” means within an acceptable error range for the particular value as determined by one of ordinary skills in the art, which will depend in part on how the value is measured or determined, i.e., the limitations of the measurement system. It is commonly accepted that a 10% precision measure is acceptable and encompasses the term “about”.

The use of the word “a” or “an” when used in conjunction with the term “comprising” in the claims and/or the specification may mean “one” but it is also consistent with the meaning of “one or more”, “at least one”, and “one or more than one”.

In the above description, an embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Moreover, it is to be understood that the invention is not limited in its application to the details of construction and the arrangement of components set forth in the following description or illustrated in the drawings. The invention is capable of other variants or embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional suitable items.

It should be understood that any one of the above-mentioned implementations of each composition, products, method, process and use of a combination of a PAC-A containing extract and a zinc-containing agent may be combined with any other of the implementations thereof, unless two implementations clearly cannot be combined due to their mutually exclusivity.

1-64. (canceled)

65. A composition for treating and/or preventing inflammation in a subject comprising A-type proanthocyanidin (PAC-A) and zinc, wherein the subject is an animal.

66. The composition of claim 65, comprising at least 0.001 wt % of PAC-A with respect to a total weight of the composition.

67. The composition of claim 65, comprising between 0.001 wt % and 10 wt % of PAC-A.

68. The composition of claim 65, comprising a PAC-A containing extract for providing the PAC-A.

69. The composition of claim 68, comprising between 0.05 wt % and 15 wt % of the PAC-A containing extract with respect to a total weight of the composition.

70. The composition of claim 68, comprising between 1 wt % and 3 wt % of the PAC-A containing extract.

71. The composition of claim 68, wherein the PAC-A containing extract is isolated from a natural source.

72. The composition of claim 68, wherein the PAC-A containing extract is provided as a PAC-A containing cranberry extract.

73. The composition of claim 68, wherein the PAC-A containing extract is a dried cranberry powder.

74. The composition of claim 65, comprising a zinc-containing compound for providing the zinc.

75. The composition of claim 74, wherein the zinc-containing compound is a zinc salt.

76. The composition of claim 75, wherein the zinc salt is selected from the group comprising zinc citrate, zinc lactate, zinc gluconate, zinc aspartate, zinc methionine, zinc oxide, zinc phosphate, zinc salicylate, and zinc chloride.

77. The composition of claim 76, wherein the zinc salt is zinc citrate.

78. The composition of claim 74, comprising at least 0.01 wt % of the zinc-containing compound.

79. The composition of claim 74, comprising between 0.01 wt % and 25 wt % of the zinc-containing compound.

80. The composition of claim 65, wherein the composition is configured for oral administration.

81. The composition of claim 65, wherein the composition is configured for topical application.

82. The composition of claim 65, wherein the composition is configured for chewable delivery.

83. The composition of claim 65, further comprising a gel base.

84. The composition of claim 65, wherein the composition is an oral care product being a mouthwash, a dentifrice, a paste, a spray, a dental chew, a gum, or a lozenge.

85. The composition of claim 65, wherein the composition is a skin care product being a gel, a lotion, a cream, or a spray that is configured for topical application to a cutaneous membrane.

86. The composition of claim 65, wherein the composition is a gastrointestinal care product being a paste, a powder, a capsule, a gummy, a liquid, a suppository or a chewable substance.

87. The composition of claim 65, wherein the composition is a paste, a powder, a gel, a liquid, or a chewable substance.

88. The composition of claim 65, wherein the subject is a dog, a cat, or a horse.

89. A method of preventing and/or treating inflammation in a subject comprising administration of a composition comprising A-type proanthocyanidin (PAC-A) and zinc, wherein the subject is an animal.
